(19) United States
(12) Patent Application Publication
(10) Pub. No.: US 2007/0196281 A1
(43) Pub. Date: Aug. 23, 2007

(54) Method and articles for remote magnetically induced treatment of cancer and other diseases, and method for operating such article

(76) Inventors: Sungho Jin, San Diego, CA (US); Thomas Pisanic, San Diego, CA (US)

Correspondence Address: PATENT DOCKET ADMINISTRATOR;LOWENSTEIN SANDLER PC, 65 LIVINGSTON AVENUE, ROSELAND, NJ, 07068, US

(21) Appl. No.: 10/584,781

(22) PCT Filed: Dec. 23, 2004
(86) PCT No.: PCT/US04/43459

§ 371 Date: May 9, 2007

Related U.S. Application Data
(60) Provisional application No. 60/533,725, filed on Dec. 31, 2003.

(51) Int. Cl. A61K 39/395; A61K 49/10; A61N 1/39

(52) U.S. Cl. 424/9.34; 424/178.1; 607/1; 977/906

(57) ABSTRACT

This invention describes unique treatment methods and innovative articles that can be placed in a human or animal body to enable controlled destruction of diseased tissue. The methods include destruction of diseased cells and tissues by magnetically controlled motion and an externally controllable drug delivery process with a capability to start and stop the drug delivery at any time, for any duration. This invention provides two approaches to diseased cell destruction, (1) magneto-mechanical disturbance of cell structure (e.g. cancer cells) for cell lysis and (2) magnetically activated drug release at local regions (e.g. tumors) from a magnetic-particle-containing drug reservoir. The invention also provides combinations of both the above treatments for dual therapy. It further combines one or both of the treatments with magnetic hyperthermia for multifunctional cell destruction therapy. The approaches can be combined with magnetic MRI for monitoring the accuracy of placement as well as for following up the cancer destruction progress and appropriate reprogramming of the magneto-mechanical therapy and remote-controlled drug release.

### CROSS REFERENCE TO RELATED APPLICATIONS

This application claims the benefit of U.S. Provisional Application Ser. No. 60/533,725 entitled “Methods and Articles for Remote Magnetically Induced Treatment of Cancer and Other Diseases, and Method for Operating Such Article”, filed by Sungho Jin on Dec. 31, 2003, which is incorporated herein by reference.

### FIELD OF INVENTION

The present invention relates to the use of magnetic particles to treat diseases and, in particular, to the use of implantable magnetic particles and remotely applied magnetic fields to treat diseased tissues and cells, such as cancers and tumors.

### BACKGROUND OF THE INVENTION

Improved methods and articles for treating diseases such as cancers and tumors are of extreme importance. According to the National Institute of Health, ˜45% of males and 39% of females will be diagnosed with some form of cancer in his/her lifetime. Beyond economics, there is no dollar value that can be placed on the emotional trauma a person goes through after being diagnosed with cancer. The economic burden of cancer to the affected individual, family and the society is tremendous. It has been estimated that the US will lose $172 billion in the year 2002 due to cancer. See R. Etzioni, et al., “The Case for Early Detection”, Nature Reviews: Cancer, Vol. 3, page 1-10, 2003. This cost arises from medical expenses, loss of work productivity due to illness, and the cost of premature death. The survival rates of cancer patients have improved significantly in the last forty years, from 30% in the 1950s to 64% in the 1990s. See L. Ries, et al, SEER Cancer Statistics Review, 1975-2000, National Cancer Institute, Bethesda, Md., 2003. The formula for the improvement in cancer survival rate has been the use of imaging technology for early detection, followed by surgical removal and possibly chemotherapy or radiotherapy. For patients who retain cancer cells in the body after surgery, the follow-up therapy, such as the chemotherapy drug delivery, is crucial for survival. Because of the severe toxicity often associated with cancer chemotherapy drugs, the practical usable dose for oral or injection administration is restricted, often to levels insufficient for cancer elimination. Targeted local delivery of cancer drugs is therefore important to enhance the therapeutic effect of chemotherapy. See “Controlled Drug Delivery” edited by K. Park, American Chemical Society, Washington DC, 1997.

It is desirable to further advance cancer treatment in an effort to improve the survival rate. Research advances in basic sciences and nanotechnology have produced a plethora of novel discoveries and treatment techniques that will be useful for engineering the next generation of cancer imaging systems. Information obtained from research findings in tissue processes (e.g., angiogenesis), cell dynamics (e.g., cell migration), and genetics can be utilized for isolating and identifying targeting molecules.

Nanometer-sized materials have unique optical, electronic, and magnetic properties that can be tuned by changing the size, shape, or composition. These materials are useful for creating new cancer therapeutic techniques and precursors for building new cancer treatment therapeutic agents. For example, targeted drug delivery using polymer-base carriers can allow higher dose cancer drugs to the localized tumor regions with minimal adverse effects on the human body. Most of the conventional drug delivery techniques depend on natural, slow diffusion of drugs from the delivery carrier or capsule, without active control in terms of delivery initiation time, duration, delivery profile, and termination time.

### SUMMARY OF THE INVENTION

This invention describes unique treatment methods and innovative articles that can be placed in a human or animal body to enable controlled destruction of diseased tissue. The methods include destruction of diseased cells and tissues by magnetically controlled motion and an externally controllable drug delivery process with a capability to start and stop the drug delivery at any time, for any duration. This invention provides two approaches to diseased cell destruction, (1) magneto-mechanical disturbance of cell structure (e.g. cancer cells) for cell lysis and (2) magnetically activated drug release at local regions (e.g. tumors) from a magnetic-particle-containing drug reservoir. The invention also provides combinations of both the above treatments for dual therapy. It further combines one or both of the treatments with magnetic hyperthermia for multifunctional cell destruction therapy. The approaches can be combined with magnetic MRI for monitoring the accuracy of placement as well as for following up the cancer destruction progress and appropriate reprogramming of the magneto-mechanical therapy and remote-controlled drug release.

### BRIEF DESCRIPTION OF THE DRAWINGS

The nature, advantages and various additional features of the invention will appear more fully upon consideration of the illustrative embodiments now to be described in detail with the accompanying drawings. In the drawings:

FIG. 1 schematically illustrates the magnetic characteristics various types of magnetic particles materials suitable for cancer treatment;

FIG. 2 represents TEM micrographs of exemplary magnetic nanoparticles suitable for cancer treatment, (a) spherical superparamagnetic Fe₃O₄ particles, (b) elongated ferrimagnetic gamma-Fe₂O₃ particles;

FIGS. 3(a), (b) schematically illustrates before and after tumor cell damage caused by rotation of elongated magnetic nanoparticles;

FIGS. 3(c),(d) schematically illustrate before and after tumor cell damage caused by oscillating lateral motion of magnetic nanoparticles;

FIG. 4. shows an apparatus for providing (a) rotational, and (b) oscillatory lateral magnetic field for particle movement;

FIG. 5. illustrates magnetically-activated, targeted cancer drug release via (a) heating, (b) applied magnetic field, (c) magnetic-induced vibration, and (d) frictional wear.

It is to be understood that the drawings are for purposes of illustrating the concepts of the invention and are not to scale.

### DETAILED DESCRIPTION OF THE INVENTION

This invention provides several approaches to diseased cell destruction, i.e., (A) magneto-mechanical disturbance of cell structure for cell lysis and (B) magnetically activated drug release at local regions from a magnetic-particle-containing drug reservoir. The invention also includes combining both of the above mechanisms (A and B) for dual therapy, as well as combining one or both of the above mechanisms (A, B or A and B) with magnetic heating of disease cells to produce hyperthermia therapy for multifunctional cell destruction. Nanoscale magnetic particles offer exciting possibilities for biomedical applications. These magnetic nanoparticles can easily be fabricated into small and controlled sizes comparable to or smaller than biological entities of interest, with their size ranging from ˜2-100 nm as compared to proteins and genes (a few to tens of nanometers) and cells (a few to hundreds of microns). The unique advantages of magnetic nanoparticles for biomedicine applications include:

i). targeting by controlled binding or tagging to specific biomolecules or tumor cells. The nanoparticles can be functionalized with a coating of bio-compatible material (e.g. polymer, dextran, silicon oxide or gold) and then conjugated with a targeting molecule such as an antibody or peptide. (see articles by M. Akeman, et al., “Nanocrystal targeting in vivo”, PNAS, Oct. 1, 2002, Vol. 99(20), p. 12617, and by O. Mylchaylyk, et al., “Glial brain tumor targeting of magnetite nanoparticles in rats”, Journal of Magnetism and Magnetic Materials, Vol. 225, p. 241-247, 2001.);

ii). mobility and navigability inside the animal or human body by externally guided magnetic fields;

iii) ability to transfer energy using applied ac magnetic field to perform localized tumor cell destruction via hyperthermia or help enhance chemotherapy with the raised temperature (see review articles by Q. A. Pankhurst et al., Journal of Physics D: Appl. Phys. Vol. 36, page R167-R181, 2003, and by P. Tartaj, et al., Journal of Physics D: Appl. Phys. Vol. 36, page R182-R197, 2003.),

iv). ability to offer contrast enhancement in magnetic resonance imaging (MRI). See the article by O. Mykhaylyk, et al. cited above.

These advantages are only beginning to be exploited for some limited biomedicine areas in recent years. In this invention, magnetic nanoparticles are distributed, targeted and manipulated to damage and destroy cancer tumor cells.

One cancer treatment using magnetic particles is magnetic hyperthermia. Hyperthermia is a therapeutic process using elevated tissue temperature for the treatment of diseased tissue such as cancer. Hyperthermia therapy consists of intentionally increasing tissue temperature to the range of ˜41 to 45° C., for a period of 30 minutes to an hour. Hyperthermia therapy kills cancer cells by various mechanisms such as protein denaturation, impairment of membrane-related functions, inhibition of the synthesis and repair of damaged DNA, proteins, and RNA, and heat damage of polysomes and microsomes.

While the biological and clinical effectiveness of hyperthermia has been proven, its utility has been restricted because of unacceptable coincidental heating of healthy tissues. The inability to localize hyperthermia to tumor regions has thus hindered its therapeutic application. Magnetic particle hyperthermia provides a solution to this problem as it ensures preferential and localized heating of only the intended target tissue (e.g. tumors with targeted/bound magnetic nanoparticles). The therapeutic efficacy of targeted magnetic hyperthermia has been clearly demonstrated by a number of investigations, e.g., using magnetic liposomes and magnetic ferrofluids via animal experiments. See P. Moroz, et al., “Magnetically mediated hyperthermia: current status and future directions”, Int. J. Hyperthermia 18, 267-284 (2002), M. Shinkai, et al., “Intracellular hyperthermia for cancer using magnetite cationic liposomes”, J. Magnetism and Magnetic Materials 194, 176-184 (1999), and A. Jordan, et al., “Presentation of a new magnetic field therapy system for the treatment of human solid tumors with magnetic fluid hyperthermia”, J. Magnetism and Magnetic Materials 225, 118-126 (2001). In addition to the magnetic hyperthermia, magnetic nanoparticles have been utilized for cancer treatment via cell separation (such as for leukemia), or magnetic guidance of cancer drugs to the tumor sites.

In accordance with the invention, the effectiveness of such treatments can be significantly enhanced by introducing additional mechanisms of cancer cell destruction. The invention introduces two additional novel mechanisms of efficient cancer cell destruction. One method involves implanting rotatable or laterally oscillating magnetic particles and applying a remote magnetic field to induce particle movement that causes mechanical disturbance and lysis of cancer cells. The other is to implant cancer-drug-carrying particles comprising magnetic nanoparticles which, on remote magnetic actuation, locally and specifically release cancer drugs to facilitate preferential damage of the cancer cells. Such an externally controllable drug delivery process offers a unique capability to start and stop the drug delivery at any time, for any durations, with any desired delivery profiles. Methods of applying such techniques are also disclosed.

In the design of magnetic nanoparticles and instrumentations for magnetic cancer treatment, it is important to understand the underlying physical behavior of magnetic nanoparticles, the movement of magnetic particles under different modes of applied magnetic field, and the mechanisms by which heat is generated in small magnetic particles by externally applied alternating current (AC) magnetic fields.

Magnetic particles move in the presence of a gradient magnetic field. Thus they can be made to rotate or oscillate laterally back and forth with time-dependent changes in field direction and magnitude. In a uniform magnetic field, particle movement is less pronounced, however, particles tend to line up along the field direction, forming a chain-of-spheres configuration, thus altering the overall shape of particle-containing systems.

In a relatively high frequency AC magnetic field, the particles are heated, thus effectuating magnetic hyperthermia treatment. Enough heat must be generated by the particles to achieve and maintain adjacent tissue temperatures of at least ˜41° C. for at least 30 minutes in order to kill the cancer cells. The mechanism of localized heat generation in magnetic hyperthermia using non-superparamagnetic particles involves mainly the magnetic hysteresis loss of energy during a magnetization-demagnetization cycle.

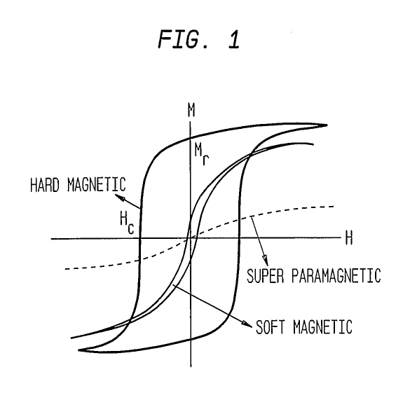

FIG. 1 is a diagram schematically illustrating the magnetic hysteresis behavior of three types of magnetic materials relevant to the magnetic cancer treatment described herein. As the applied magnetic field (H) is increased from zero to a finite value and then reduced again in both positive and negative field directions, a magnetic material exhibits a magnetization M-H loop, the characteristics of which depend on the type of magnetic material involved. Hard magnetic materials have high coercivity (H_{c}) and remanent induction (Mᵣ). They exhibit a large hysteresis loop behavior as illustrated in FIG. 1. The magnetically hard material is difficult to magnetize, requiring a strong applied field of e.g., 10-1000 KA/m (˜120-12,000 gauss) to be fully magnetized. But once magnetized, it tends to retain magnet characteristics (high Mᵣ) even after the applied field is removed (H=0). Soft magnetic materials are much easier to magnetize or demagnetize using a relatively weak magnetic field of ˜10-100 KA/m (12-120 gauss), but the value of remanent induction is small. Superparamagnetic materials have extremely small particle sizes of typically ˜10 nm or less in diameter (depending on the anisotropy of the material), exhibit no overall magnetic hysteresis and no remanent induction because of the magnetic moment fluctuation by thermal energy at a given temperature.

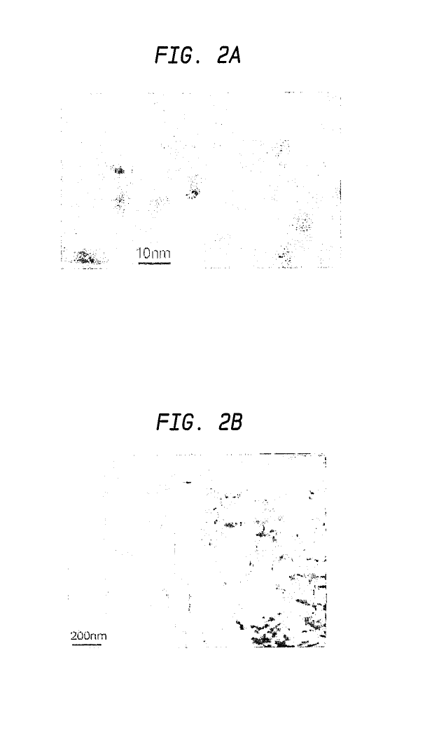

FIGS. 2(a) and 2(b) are transmission electron micrographs (TEMs) of exemplary magnetic nanoparticles suitable for cancer treatment. FIG. 2(a) depicts synthesized superparamagnetic Fe₃O₄ (magnetite) particles . The magnetic susceptibility (the slope of the magnetization curve) and magnetic strength of superparamagnetic particles are significantly lower than those for the soft magnetic materials. Because of their zero or small remnant induction, superparamagnetic particles and multi-domain soft magnetic particles usually do not agglomerate easily, which is desirable for magnetic hyperthermia or magnetic MRI applications. The hard magnetic particles tend to easily agglomerate due to their high remnant magnetization. Coated magnetic particles are less prone to agglomeration because of inter-particle gaps. FIG. 2(b) depicts ferromagnetic gamma-Fe₂O₃ particles elongated in one particle dimension.

The magnetic hysteresis behavior of magnetic particles when exposed to a time-varying externally applied AC magnetic field produces magnetically induced heating. The amount of hysteresis-induced heat generated per unit volume is proportional to the frequency of the applied field multiplied by the area of the hysteresis loop of the material (FIG. 1). Magnetically hard material with high coercive force, high remnance and large hysteresis loss can generate more heat. However, magnetically soft materials may have an operational advantage because of the ease of reaching a high magnetization state with a relatively low, practically available AC field. Also, the tendency of undesirable particle agglomeration with high coercivity materials can cause a problem in dispersion and targeted distribution of hard magnetic nanoparticles to the desired site. Superparamagnetic particles are ideal in this sense as there is no remanent magnetism in the absence of field to cause magnetic agglomeration.

From a practical point of view, the frequency and strength of the externally applied AC magnetic field that can be employed to generate the appropriate level of heating in a human is limited by deleterious physiological responses to high frequency magnetic fields. Such responses include undesirable stimulation of peripheral and skeletal muscles, possible cardiac stimulation and arrhythmia, and non-specific inductive heating of tissue. [See articles by J. R. Oleson, et al., “Hyperthermia by magnetic induction: experimental and theoretical results for coaxial coil pairs”, Radiat. Res. 95, 175-186 (1983), and by J. P. Reilly, et al., “Principles of nerve and heart excitation by time-varying magnetic fields”, Ann. New York Acad. Sci. 649, 96-117 (1992).] A safe range of frequency and amplitude of AC field is approximately ˜0.05-1.2 MHz in frequency and ˜0-15 kA/m in field strength (equivalent to ˜0-180 gauss). The frequency and magnitude of the required field for efficient magnetic hyperthermia heating depends on several factors, such as the amount of magnetic nanoparticle material introduced, the nature and size of the magnetic material used, whether the nanoparticles are directly injected to the local tumor region, and the efficiency of tumor-targeted binding. A rough estimate is that several milligrams of magnetic material concentrated in each cubic centimeter of tumor tissue are appropriate for magnetic hyperthermia in human patients. [See the article by Q. A. Pankhurst, et al., cited earlier.]

Candidate magnetic nanoparticle materials suitable for the invention articles can be selected from ferromagnetic or ferrimagnetic materials with: i) generally larger multi-domain particles; ii) single-domain size particles (˜8-30 nm size); or iii) smaller, superparamagnetic particles (˜2-15 nm size). These particle sizes are sufficiently small to allow effective delivery to the site of the cancer, either via encapsulation in a larger moiety or suspension in a carrier fluid. Nanoscale particles can be coupled with antibodies to facilitate targeting on an individual cell basis. The mechanism of heat generation associated with each type of materials can be different, offering unique advantages and disadvantages. The iron oxides magnetite (Fe₃O₄) and maghemite (γ-Fe₂O₃) are the most commonly used materials due to biocompatibility and suitable magnetic properties. Other highly magnetic nanoparticles such as iron, nickel, cobalt, and magnetically soft ferrites such as Co-ferrite, Mn—Zn ferrite and Ni—Zn ferrite may also be used.

For in vivo applications the magnetic particles must be coated with a biocompatible material such as various bio-complete polymers, dextran, SiO₂, or gold, during or after the synthesis process to prevent the formation of large aggregates. Biocompatible polymer or SiO₂ coatings also permit relatively easy binding of therapeutic drugs to the magnetic particles via covalent attachment, adsorption or entrapment. See B. Denizot, et al., “Phosphorylcholine Coating of Iron Oxide Nanoparticles”, J. Colloid Interface Sci. 209 66 (1999), and a book by U. Hafeli et al., Scientific and Clinical Applications of Magnetic Carriers, New York: Plenum, 1997.]. The main advantages of using nanoparticle sizes of less than 100 nm are their higher effective surface areas for easier attachment of ligands, lower sedimentation rates (high dispersion stability) and improved diffusion in tissues.

The magnetic nanoparticles for magneto-mechanical cell destruction or remote magnetic actuation for time-controllable drug delivery can be placed into the tumor by one or more of four mechanisms: 1). By injecting the magnetic nanoparticles into the blood vessel and allowing the tumor cell targeting to take place (e.g., by attached peptide or antibody on the particle surface); 2). By allowing the cells to naturally engulf (endocytosis) the particles, 3) By magnetically navigating/guiding the particles, e.g., dragging them using external permanent magnetic, or 4) By magnetofection forcing the particles through the cell walls into intracellular regions, for example using a gradient magnetic field. For accuracy of targeted cancer cell destruction or drug delivery, the positioning of magnetic nanoparticles at or near the tumor location and their distribution is desirably confirmed before the magneto-mechanical cell destruction is applied. Either optical or MRI imaging can be utilized.

(A). Tumor Cell Destruction using Magneto-Mechanical Agitation

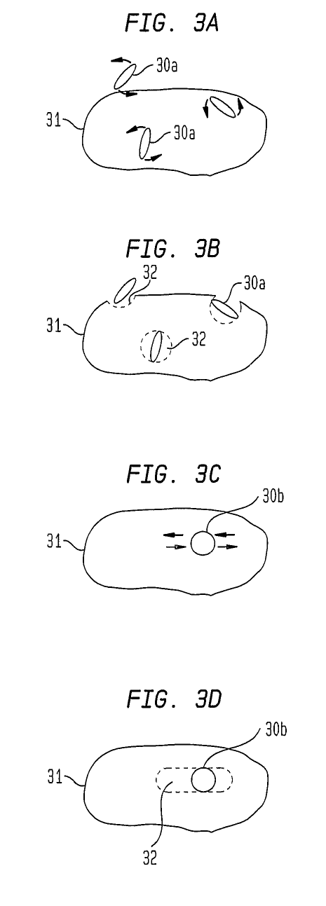

This approach uses magnetic nanoparticles coated with a biocompatible material such as dextran or silica, and then functionalized with peptide or antibody on the magnetic nanoparticle surface. The peptide or antibody on the magnetic particles allows targeting of the particles onto cancer cell surfaces. Alternatively, the particles can be moved toward and placed inside of the cancer cells by endocytosis or by an intentional application of gradient magnetic field (e.g., ˜100-10,000 Gauss/cm gradient) which can force the magnetic nanoparticles to move along the gradient direction passing into the cells on their way. Referring to FIG. 3(a)-(d), the magnetic nanoparticles 30A, 30B on or inside the tumor cells 31 are magnetically moved in a controlled manner to induce magneto-mechanical damage 32 of tumor cells 31. By utilizing elongated magnetic nanoparticles 30A, such as maghemite (γ-Fe₂O₃) shown in FIG. 2(b), and applying a rotational magnetic fields such as by sequential actuation of remote electromagnets, the particles can be made to rotate at appropriate frequencies. Such a nano-blender type mechanical motion can disrupt the structure of regions 32 in the tumor cell, as illustrated in FIG. 3(b). An alternative way of producing cell mechanical damage is to use a laterally oscillating gradient magnetic field to laterally oscillate magnetic particles 30B causing cell damage 32 as illustrated in FIG. 3(d).

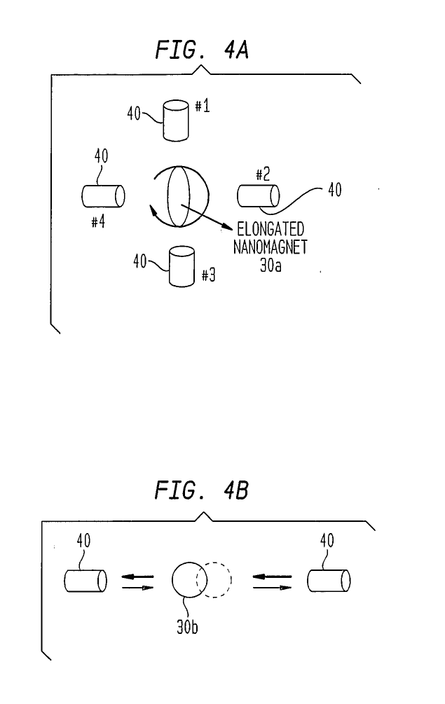

FIGS. 4(a) and 4(b) schematically illustrate the mechanisms of moving the nanoparticles to damage or destroy diseased cells. In FIG. 4(a) a plurality of electromagnets 40 (preferably external to the patient) are disposed at positions circumferentially around elongated nanomagnet 30A. When the electromagnets are sequentially activated, as through the sequence #1, #2, #3, #4, then nanomagnet 30A will rotate, e.g. clockwise to destroy cell components in its locus of rotational movement.

In FIG. 4(b) a plurality of electromagnets 40 (preferably external) are disposed on opposite sides of nanomagnet 30B and driven in alternation so that the nanomagnet is driven back and forth laterally, inflicting mechanical damage to cell components in its locus of oscillatory movement.

Another cancer treatment involves a combination of magneto-mechanical cell destruction and magnetic hyperthermia. The same magnetic nanoparticles targeted and attached to the cancer cells can be utilized for both mechanical movement and heating. This combination further enhances the overall probability of complete cancer elimination. Incomplete cancer cell destruction is often not an acceptable solution in cancer treatment because of cancer recurrence when even a small number of cancer cells remain.

A proper magnetic field magnitude, frequency, and field direction can in principle be formulated to achieve the goals of both magneto-mechanical cell destruction and magnetic hyperthermia simultaneously. However, a preferred treatment desirably consists of two steps, for example, a step of applying a rotating or laterally oscillating field within a somewhat lower frequency range of e.g., 1 Hz-500 KHz for the magneto-mechanical cell destruction, and then a second step of applying a stationary, higher frequency field (e.g., 1 KHz-5 MHz) for magnetic hyperthermia. The two steps can be applied in series or they can be intermixed, as for example, alternately applying 10 minutes of each step.

The instrumentation suitable for magnetic hyperthermia therapy consists of a high frequency AC solenoid with adjustable frequency and amplitude in the range of ˜0.1 KHz-50 MHz (preferably 1 KHz-5 MHz) in frequency and ˜0-1500 KA/m (0-180 gauss), preferably 1-15 KA/m (12-180 gauss) in field strength. The use of a soft magnetic, high saturation, high-permeability core such as iron, Co—Fe, permalloy (Ni—Fe alloy), Ni—Zn ferrite or Mn—Zn ferrite is preferred for field amplifying purposes. The tissue temperature rise during the AC field magnetic hyperthermia can be accurately measured using a non-metallic, optical fiber thermometer.

(B). Magnetic Drug Delivery

Therapeutic drugs for critical applications such as chemotherapies on tumors are typically administered in a non specific way. This is one of the main disadvantages of the current processes as the cytotoxic drug causes deleterious side-effects as it indiscriminately attacks normal, healthy cells. If the drug treatments could be localized, e.g. to the specific tumor site, very potent doses of effective agents could be utilized with minimal side effects.

In magnetically targeted drug therapy, according to the invention, a cytotoxic drug can be 1) attached onto the surface of functionalized and properly conjugated biocompatible magnetic nanoparticle carrier, 2) included inside a porous polymer containing magnetic particles in the pores, or 3) encapsulated in magnetic liposomes. Some of these inventive drug/carrier complexes, such as biocompatible ferrofluids, can be injected into the patient's circulatory system, and the particles can either self-target the tumor cells due to the antibody conjugation added on their surface, or can be guided and kept in place by external, high-gradient magnetic fields. Alternatively, they can be needle-injected into the tumor area followed by self-targeting, endocytosis or magnetofection. Once the drug/carrier is concentrated at the targeted organ, the drug can be released by a number of approaches such as via enzymatic activity, changes in physiological conditions such as pH, osmolality, or local temperature. Targeted drug delivery using these principles have been widely used for non-magnetic drug delivery. See the book on drug delivery by. K. Park cited earlier. Not much work has been done regarding the use of magnetic field for controlled drug release, although magnetic guidance to bring a drug toward an intended organ has been demonstrated. See C. Alexiou, et al., “Locoregional cancer treatment with magnetic drug targeting”, Cancer Res. 60, 6641-8 (2000).

Generally, the magnetic particles in this invention are coated by a biocompatible material such as PVA or dextran, or inorganic coatings such as silica or gold. The coating protects the magnetic particle from the surrounding environment and also facilitates functionalization by attaching to carboxyl groups, biotin, avidin, carbodi-imide and other molecules These functional group molecules can act as attachment points for cytotoxic drugs or target antibodies to the carrier complex.

Some success in targeted delivery of magnetic drug carriers has recently been reported with human and animal experiments. A total remission of sarcomas was achieved in rats using magnetically targeted cytotoxic drugs, doxonibicin, implanted in rat tails. See K. J. Widder, et al., “Selective targeting of magnetic albumin microspheres containing low-dose doxorubicin—total remission in Yoshida sarcoma-bearing rats”, Eur. J. Cancer Clin. Oncol. 19 135-139 (1983). A similar technique has been employed to target cytotoxic drugs to brain tumors. It was demonstrated that 10-20 nm magnetic particles were effective at targeting these tumors in rats. Electron microscopy analysis showed that magnetic carriers were actually present in the interstitial space in tumors. See S. K. Pulfer, et al., “Distribution of small magnetic particles in brain tumor-bearing rats”, J. Neuro-Oncol. 41, 99-105 (1999). Promising results related to magnetic targeting in humans were also reported. A Phase I clinical trial reported by Lubbe et al., “Physiological aspects in magnetic drug-targeting”, J. Magnetism and Magnetic Materials 194, 149-155 (1999), demonstrated that drug-targeting with a ferrofluid (1% of the blood volume, Fe₃O₄ magnetic particle size of 100 nm, coated with a starch derivative) with the magnetic particles bound to “epirubicin” cancer drug, caused complete remissions of human colon as well as renal cancer. The reversible heteropolar binding of the drug epirubicin from the magnetic particles allowed the diffusion through the vessel wall into the tumor interstitial space. In addition, the article reported that the ferrofluid was successfully directed to the advanced sarcomas tumors without associated organ toxicity.

It is noted that these prior art techniques primarily deal with magnetic navigation and magnetic hyperthermia treatment, i.e., magnetic-field-assisted guiding of nanoparticle drug carriers and holding them in place for drug delivery, rather than magnetically actuated drug release.

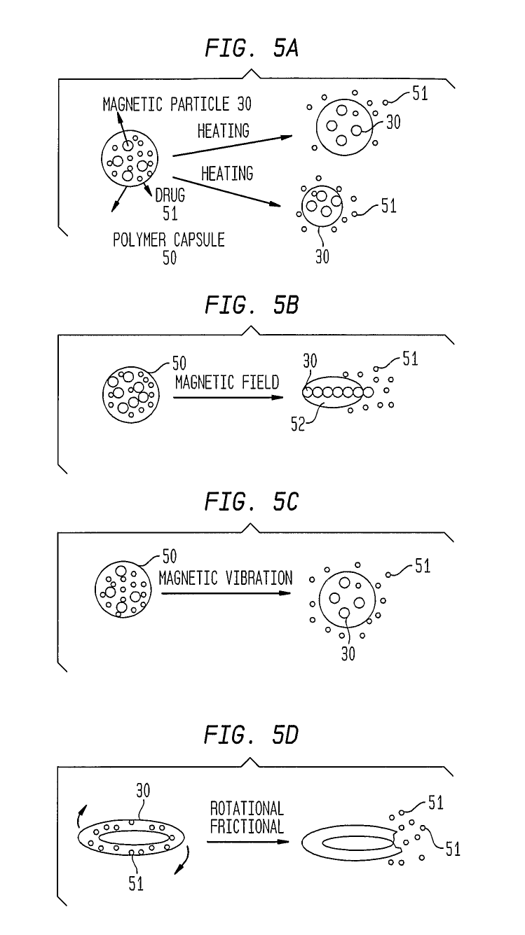

Magnetically actuated drug release, according to the invention, offers programmable, remotely controlled drug release. This provides the ability to administer the drug therapy—i) at any time, ii) for any duration, iii) at any programmable dose strength and release profile, iv) any-time termination of drug release. The technique can also be utilized for delivery of other drugs to human or animal organs for cure or alleviation of other non-cancer diseases or pains. Some exemplary approaches to the magnetically actuated drug release are schematically illustrated in FIG. 5.

i). In FIG. 5(a), a capsule 50 contains magnetic particles 30 and cancer drug(s) 51. The drugs 51 can be released via magnetic heating, e.g. during hyperthermia. It is well known that there are many temperature sensitive polymers and hydrogels that can melt, swell or shrink to release drugs. See Biorelated Polymers and Gels—Controlled Release and Applications in Biomedical Engineering, T. Okano edited, Academic Press, Boston 1998, p. 93. For example, poly(N-isoporpylacrylamide)(NIPAAm) is one of the representative temperature-sensitive polymers with a lower critical solution temperature (LCST) of ˜32° C. Such capsules are made to contain cancer drugs 51 and magnetic nanoparticles 30 together (or side by side in two adjacent chambers in a capsule), for example, using emulsion techniques. The drug can be dissolved in an aqueous solution or biocompatible solvent, in the form of deformable jelly, or in the form of nanoparticles mixed in the solidified polymer. The drug-containing nano-capsules, e.g., 20-2000 nm size, having a spherical, pancake or elongated rod shape, are then placed inside a human or animal body, either through injection into the blood stream, into the tumor or into the tumor region. The magnetic particles containing the desired cancer drugs are then placed inside the tumor by either injecting them into the blood vessel and allowing the tumor cell targeting to take place (e.g., by attached peptide or antibody on the particle surface), by letting the cells naturally engulf (endocytosis) the particles, by magnetically navigating/guiding the particles, e.g., dragging them using externally sweeping permanent magnets, or by using magnetofection forcing the particles to pass through the cell walls into intracellular regions, for example using a gradient magnetic field. For accuracy of targeted drug delivery, the positioning and distribution of the magnetic nanoparticles at or near the tumor location is desirably confirmed, e.g., by MRI imaging, prior to delivery of the drug. To effect delivery an external magnetic field is applied so that the magnetic particles are locally heated, which in turn heats the temperature-sensitive polymer as well as the solution (such as saline, simulated body fluid solution, or other organic or inorganic solvent if the drug is already dissolved in the solution) in the polymer nanocapsule. The heating and expansion of the solution can cause the solution to leach out. Alternatively, the contraction of the polymer capsule diameter can cause the drug to leach out.

ii). FIG. 5(b) shows magnetic alignment and puncturing of capsule wall. When a DC or AC magnetic field is applied (or removed), magnetic particles 30 inside a drug-containing capsule 50 move and rearrange themselves to reduce the overall magnetostatic energy. Either formation of a long chain-of-spheres 52 or agglomeration and squeezing action of magnetic particles occurs depending on the initial state of particle arrangement, magnetic properties of the particles, and viscosity of the drug-containing matrix. The chain formation 52 elongates the length, and can apply enough stress to squeeze out a liquid drug 51 the from polymer pores, or to puncture the capsule wall to release the drug 51.

iii). FIG. 5(c) shows how a high frequency AC field can induce magneto-mechanical vibration, which can cause a capsule 50 to release a mechanically retained drug 51 in a nanocomposite particle mix or slurry of magnetic nanoparticles 31, liquid-, jelly-, or particle-shaped polymer material. The cancer drug 50 can be in the form of either a drug solution, drug jelly or drug nanoparticles.

iv). FIG. 5(d) illustrates the magnetically induced release of drugs by wearing away of particles 30. Elongated drug-carrying magnetic particles (or capsules) 30, 50 can encounter significant frictional force on its ends if a high-speed rotating or oscillating magnetic field is applied. When the tip of the elongated particles (or capsules) containing the drug 51 breaks off or wears away, the drug can be released from the ends.

The inventive magnetic nanoparticle cancer therapy can also be combined with magnetic-particle MRI (magnetic resonance imaging). The magneto-mechanical cell destruction treatment, the magnetic hyperthermia treatment, or the combination therapy of both can be combined with the magnetic-particle MRI for imaging and confirmation of the accuracy of magnetic therapy particles placement.

MRI relies on the counterbalance between the extremely small magnetic moment on a proton, and the very large number of protons present in biological tissue, allowing a measurable effect in the presence of high magnetic Fields. See articles by M. Browne and R. C. Semelka, MRI: Basic principles and applications, Wiley, New York 1999, and by J. D. Livingston, Driving Force: The Natural Magic of Magnets, Harvard Univ. Press, Cambridge, Mass. 1996.

The presence of very fine superparamagnetic or magnetic particles can enhance the contrast in MRI. Such a magnetic MRI imaging offers the advantage of high spatial resolution displaying contrast differences between tissues. In search of an effective contrast agents that will enhance and widen its diagnostic utility, there has been increasing interest and clinical diagnosis applications of contrast agents like dextran magnetite for MRI. See M. Shinkai, “Functional magnetic particles for medical application”, J. of Biosci. and Bioeng. 94(6), 606-613 (2002), Compared with paramagnetic ions, superparamagnetic iron oxide particles have higher molar relaxivities, and, when used as blood pool and tissue-specific agents, may offer advantages at low concentrations. Tumor-targeted magnetic MRI studies have also been conducted, demonstrating significant enhancement of MRI image contrast. See the article by O. Mykhaylyk, et al. cited earlier, an article by D. K. Kim, et al., “Characterization and MRI study of surfactant-coated superparamagnetic nanoparticles administered into the rat brain”, J. Magnetism and Magnetic Materials 225, 256-261 (2001), and an article by C. Alexiou, et al., “Magnetic mitoxantrone nanoparticle detection by histology, X-ray and MRI after magnetic tumor targeting”, J. Magnetism and Magnetic Materials 225, 187-193 (2001).

The present invention is also applicable for various types of medical treatments not related to the cancer treatment. For example, the unique advantages of the inventive magnetic remote drug delivery system, i.e. the capability to remotely administer the drug therapy from outside the body—i) at any time, ii) for any duration, iii) at any programmable dose strength and release profile, iv) any-time termination of drug release, can be utilized for delivery of other drugs to human or animal organs for curing or alleviating of various diseases or symptoms, for example, delivery and controlled release of diabetes medications (insulin), gastrointestinal drugs, cardiovascular medicines, control drugs for brain functions and abnormal behavior, muscle control medicines, pain killers, antibiotics, gene therapy. The presence of magnetic particles can also be utilized to locally raise the temperature of the released drugs, via a magnetic hyperthermia process, to accelerate the therapeutic efficiency of drug-cell interactions.

It is understood that the above-described embodiments are illustrative of only a few of the many possible specific embodiments which can represent applications of the invention. Numerous and varied other arrangements can be made by those skilled in the art without departing from the spirit and scope of the invention.

What is claimed is:

1. A method of treating diseased cells in a human or other animal body comprising the steps of:
providing particles comprising one or more nanoparticles of magnetic material, the particles optionally including medication;
introducing the particles into the body;
directing the particles into or adjacent the diseased cells; and
applying a magnetic field to the magnetic nanoparticles to treat the diseased cells by magnetically induced motion of the nanoparticles or by magnetically induced release of the medication.

2. The method of claim 1 wherein the application of the magnetic field treats the diseased cells by moving the nanoparticles to damage or destroy the cells.

3. The method of claim 1 wherein the particles include medication and the application of the magnetic field effects delivery of the medication to the diseased cells.

4. The method of claim 3 wherein the medication comprises a cytotoxin.

5. The method of claim 1 wherein the particles comprise magnetic nanoparticles coated with bio-compatible material.

6. The method of claim 5 wherein the bio-compatible material comprises a material selected from the group consisting of bio-compatible polymers, dextran, silicon oxide and gold.

7. The method of claim 1 wherein the particles are introduced into the body by injection.

8. The method of claim 1 wherein the particles are directed into or adjacent the diseased cells by one or more targeting molecules attached to the particles.

9. The method of claim 8 wherein the targeting molecules comprise an antibody or a peptide.

10. The method of claim 1 wherein the particles are directed into or adjacent the diseased cells by magnetic navigation.

11. The method of claim 1 wherein the particles are directed into the diseased cells by magnetic transfection.

12. The method of claim 1 wherein the particles are attached to molecules to stimulate endocytosis of the particles by the cells.

13. The method of claim 1 wherein the nanoparticles of magnetic material are elongated along one dimension and the magnetic field rotates the nanoparticles to mechanically damage diseased cells.

14. The method of claim 13 wherein the magnetic field is an AC magnetic field at a frequency in the range 1 Hz to 500 Hz.

15. The method of claim 1 wherein the magnetic field laterally oscillates the nanoparticles to mechanically damage diseased cells.

16. The method of claim 1 wherein the particles comprise a heat sensitive reservoir of medication and the application of the magnetic field to the nanoparticles provides heat to effect delivery of the medication.

17. The method of claim 16 wherein the magnetic field is an AC magnetic field at a frequency in range 1 KHz-5 MHz.

18. The method of claim 1 wherein the particle comprises a reservoir of mechanically retained medication and the application of the magnetic field to the nanoparticles provides mechanical damage to the reservoir to effect delivery of the medication.

19. The method of claim 18 wherein the magnetic field provides mechanical damage to the particle by moving it to wear away portions of the particle.

20. The method of claim 18 wherein the magnetic field provides mechanical damage to the particle by moving nanoparticles within the particle.

21. The method of claim 1 wherein the step of applying a magnetic field to the magnetic nanoparticles comprises an application of a magnetic field to mechanically damage the diseased cells by rotating or oscillating the nanoparticles and an application of a second magnetic field to thermally damage the diseased cells by heating the nanoparticles.

22. The method of claim 1 wherein the particles comprise a heat sensitive reservoir of medication and the application of the magnetic field to the nanoparticles provides heat to effect delivery of the medication and to damage the diseased cells by heat.

23. The method of claim 1 wherein the particles comprise a reservoir of mechanically retained medication and the application of the magnetic field comprises an application of a first magnetic field to mechanically damage to the reservoir to effect delivery of the medication and an application of a second magnetic field to heat the nanoparticles for thermal damage to the diseased cells.

24. The method of claim 1 further comprising the step of confirming the adjacency of the particles to diseased cells or tissue prior to applying the magnetic field.

25. The method of claim 22 wherein the adjacency is confirmed by MRI imaging.

26. An article for treating diseased cells in human or other animal bodies comprising:
a particle comprising one or more nanoparticles of magnetic material, the particle coated with biocompatible material and attached to a targeting molecule for targeting diseased cells.

27. The article of claim 24 wherein the nanoparticles are elongated in one dimension.

28. The article of claim 24 wherein the particle further comprises a reservoir of medication that can be released by heat or by mechanical damage to the particle.
